(19) United States
(12) Patent Application Publication
(10) Pub. No.: US 2019/0076228 A1
(43) Pub. Date: Mar. 14, 2019

(54) LARGE ANIMAL TREATMENT TABLE

(71) Applicant: Bernd Kober, Schwetzingen (DE)

(72) Inventors: Bernd Kober, Schwetzingen (DE); Tim KOWALEWSKI, Erlensee (DE)

(21) Appl. No.: 16/124,478

(22) Filed: Sep. 7, 2018

(30) Foreign Application Priority Data
Sep. 12, 2017 (EP) 17190595.3

(51) Int. Cl. A61D 3/00; A61B 6/03; A61B 6/04

(52) U.S. Cl. CPC A61D 3/00; A61B 6/0457; A61B 6/035

(57) ABSTRACT

A large animal treatment table includes a movable base frame having front and rear portions separated by a transverse center plane, the front portion including right and left front frame parts spaced by a first gap. The table also includes a lift disposed entirely rearward of the transverse center plane. A tabletop is connected to the lift and has a front portion and a rear portion separated by the center plane. The front portion of the tabletop is cantilevered over the first gap such that, in a side view, the base frame and the lift and the tabletop have a C-shaped profile. The base frame and the tabletop are also configured such that the tabletop is movable over a table of a treatment device and such that an emitter head of an imaging device is rotatable around the cantilevered front portion.

### CROSS-REFERENCE TO RELATED APPLICATIONS

This application claims priority to European patent application no. 17 190 595.3 filed on Sep. 12, 2017, the contents of which are fully incorporated herein by reference.

### TECHNICAL FIELD

The present disclosure relates generally to mobile treatment (procedure) tables for veterinary medicine, which are also usable for large animals such as, for example, horses, and on which an animal is supported on a height-adjustable tabletop for examination, diagnostic and/or treatment purposes.

### BACKGROUND

Large animal treatment tables of the above-mentioned type are required for the universal (overall) diagnosis and treatment of small and large animals. In these situations, the animals are supported on a tabletop in order to have access to the animal body for carrying out examination and/or diagnostic and/or treatment and/or other medical procedures. The use of the phrase “treatment table” herein is intended to cover such tables whether they are used in connection with an actual medical treatment or merely for purposes of examination, diagnosis or other types of medical procedures.

Thus, for example, a large animal treatment table is distributed under the name Haico PRO3 Equine Operating Table, the tabletop of which is height adjustable and is tiltable and inclinable in four directions. The load capacity is 1,300 kg. Support extensions are attachable laterally to the tabletop. A recess is provided in a central part of the tabletop in order to increase the freedom of movement for an operator. All support parts can be removed. A head part can be laterally displaced. Due to the central lifting device, this treatment table is not combinable with other known treatment tables. Thus in particular this known large animal treatment table does not allow radio-oncological treatments using computer tomography to be performed and does not allow usage of medical devices such as medical linear accelerators having a rotatable, orbital or otherwise moveable emitter head.

A movable examination table for human medicine, which is not designed or suitable for large animals, is known from DE 10 2010 064 209 A1 and U.S. Pat. No. 8,370,975. These references disclose a movable examination table that comprises a mobile platform for supporting a human, a support attached under the mobile platform and used for supporting the mobile platform, and a guide rail disposed under the mobile platform. The examination table further comprises a transport device that can be used for driving the support to move on the guide rail. In particular, in an emergency, this examination table should allow the mobile platform on the examination table to be quickly separated from the transport device and to be returned into the initial position after the emergency has been remedied.

A mobile examination table, which is also known from human medicine, is distributed under the name Roesys X Mobil by the company Medizintech St. Egidien GmbH. This mobile examination table is also suitable only for maximum patient weights up to 250 kg and for this reason alone is not suited for large animals.

### SUMMARY OF THE DISCLOSURE

There is an increasing demand for large animal treatment (procedure) tables that are usable not only for examination- and treatment-purposes, but also permit operations (surgeries), radio-oncological treatments, radiation therapy, etc. to be performed on large animals.

The technical problem underlying the present disclosure thus involves providing a large animal treatment table that allows examinations, operations (surgeries) and/or treatments to be carried out on large animals, e.g., using known X-ray and radiation therapy devices such as, e.g., medical linear accelerators (“medical LINACs”) and computer tomography devices (“CT scanners”).

In one aspect of the present disclosure, a large animal treatment table is disclosed that comprises a movable base frame, a lifting device and a tabletop. The base frame includes, in the longitudinal axis direction, a front end and a rear end, and at least in the region of the front end comprises a divided chassis made up of a right-hand chassis part and a left-hand chassis part spaced from the right-hand chassis part in order to provide a free space (open space) below the tabletop, which free space is accessible from the front end. The lifting device comprises a lower support, using which the lifting device is disposed in the region of the rear end of the base frame, and an upper support. The tabletop is connected to the upper support of the lifting device such that it has a freely protruding (cantilevered) table region, and in a side view the base frame, the lifting device, and the support plate form an asymmetrical, essentially C-shaped outline. Owing to the divided chassis and the asymmetric arrangement of the lifting device, the tabletop is movable over an existing table of a conventional treatment or diagnosis device, such as, for example, a computer tomography device. Additionally or alternatively, owing to the disclosed design of the large animal treatment table, a treatment head of a medical linear accelerator can be rotated about the protruding (cantilevered) table region.

The above-described aspect is based upon the fact that the basic components of the large animal treatment table are configured such that, despite the considerable loads that must be supported, even a large animal is introducible into known X-ray- or computer-tomography-devices in order to be able to carry out examinations and/or treatments on the large animal with the aid of these devices. By offsetting the lifting device toward the rear end and providing a divided chassis configuration, the large animal treatment table is movable over tables fixedly connected to the examination/treatment devices. Despite the heavy loads to be supported, the C-shaped design has surprisingly proven to be suitable. Owing to the design of the chassis that is divided in plan view, at least in the front region it is also possible to also carry out on large animals so-called rotational- and stereotactic-irradiation using medical linear accelerators, since the treatment (emitter) head of the medical linear accelerator can be rotated by 360° about the large animal while lying on the treatment table and/or can be fixed (held stationary) at any rotational position around the large animal. Since the tabletop of the disclosed treatment table can be lowered to the minimum height of the existing table of the treatment device or diagnostic device, it is possible for the first time to irradiate animals having a large body circumference, such as, for example, horses, e.g., using a closely placed electron waveguide.

The tabletop is configured to be as rigid as possible for supporting the heavy loads while taking into account the protruding (cantilever) design. The tabletop is preferably composed, at least partially and/or in the front region, of materials that have high rigidity and low radiation absorption. Suitable materials are, for example, carbon fiber reinforced plastics (polymers) (often abbreviated as “CRP” or “CFRP”).

Because the disclosed large animal treatment table is movable over tables of known medical devices with the large animal located (lying) thereon, known computer tomography (CT) devices, X-ray devices, and conventional medical linear accelerators can also be readily used with large animals. Until now, to use such medical devices on large animals, the stationary table of the medical device had to be removed. However, an exchange of a table of, e.g., an irradiation device or computer tomography device, is possible only with very great effort and is extremely cost-intensive.

In an exemplary embodiment of a large animal treatment table according to the present disclosure, the lifting device, viewed in the longitudinal direction, comprises a left lifting part and a right lifting part, respectively, between which a free (open or empty) space is present. The free space between the left and right lifting parts allows the tabletop to be movable over an existing treatment table, for example, of a computer tomography device (CT scanner), and specifically so far that the lifting parts come to rest to the right and left of the existing table. Consequently the tabletop is movable, for example, very deep into a gantry of a computer tomography device.

In another exemplary embodiment, a motor is preferably provided, using which both the right and the left lifting part are adjustable in height using a transmission. For this purpose, for example, a front bearing of the lifting parts is connected to a threaded bush via a common threaded spindle. By rotating the threaded spindle, the distance between the front bearing and the threaded bushing changes; in accordance with this changing distance, a lowering or lifting of the lifting parts is effected. The threaded spindle is preferably rotated by an electric or hydraulic motor. A manual crank can preferably also be provided, using which a user may manually adjust the height in the event of a motor failure.

In a further exemplary embodiment, the right and left lifting part each comprise a scissor mechanism. A constructively simple, cost-effective and simultaneously highly load-bearing design of the lifting device is thus possible in such an embodiment. Furthermore the treating veterinarian can also comfortably sit on the rear tabletop end by placing his/her legs between the two lifting parts.

To improve the stability and/or the tilting resistance of a large animal treatment table according to the present disclosure, the base frame may preferably have a greater width at least at the front end than at the rear end. In such an embodiment, the accessibility of certain body regions of the large animal lying on the tabletop is made easier for an operator or veterinarian.

In a further exemplary embodiment the tabletop is inclinable. This is advantageous for certain examinations, operations (surgeries) and/or treatments.

The inclination of the tabletop is preferably adjustable by using a rigid locking element that is rotatably connectable at one end to the upper support in the region of the table rotation axis and at the other end to the base frame. Owing to the simple and cost-effective design of this aspect of the present disclosure, the tabletop automatically inclines/declines about a transverse axis when the lifting device is being lowered/raised. Thus no separate drive is required for adjusting the inclination. Due to the length of the locking element and the possible connecting points to the support and the base frame, the maximum inclination as well as the inclination rate when the tabletop is lowered are determinable. The inclination rate indicates by how many degrees the tabletop inclines at a defined lowering height; i.e., for example, an inclination by 10° is achieved with a lowering of 200 mm.

The rigid locking element is preferably configured as a pivotable bracket or in the form of at least one bar whose ends are configured to be pluggable or suspendable at corresponding points on the base frame and/or on the upper bearing. A cost-effective and simultaneously reliable inclination adjustment is thus realizable with such an embodiment of the present disclosure.

For the purpose of adjusting the inclination of the tabletop, the upper support of the lifting device is preferably rotatably supported with the tabletop about a transverse axis at a rear upper bearing and slidingly or rollingly supported with the tabletop at a front upper bearing.

Furthermore, for example, the lower support of the lifting device can also be rotatably supported about a transverse axis with the base frame at a rear bearing and slidingly or rollingly supported on the base frame at a front bearing. In the present specification, “slidingly supported” is intended to comprise not only classic sliding connections, but also rolling- or rolling-element-bearing assemblies.

In an exemplary, cost-effective embodiment of the above-described large animal treatment table, the right-hand chassis part and the left-hand chassis part are each provided with rollers, and preferably, at least in the region of the front end, define a free opening (clearance) having a clear inner width of at least 700 mm. This facilitates movement of the large animal treatment table over or along existing tables of medical examination- or treatment-devices of the types mentioned hereinabove and -below.

At least in the region of the front end, the base frame preferably has a maximum height over the ground of approximately 150 mm.

In order to provide a suitable working height for a veterinarian, but also to be able to carry out all conceivable treatments using existing medical devices, the tabletop is, for example, lowerable to a minimum height of approximately 700 mm and/or raisable to a maximum height of approximately 1400 mm. In such a design of a large animal treatment table according to the present disclosure, it is also possible to provide a lifting device that allows this low lowering of the tabletop despite the heavy load. This is advantageous in particular when irradiation of skin tumors using electrons is to be carried out on the large animal. In this type of electron irradiation, an electron waveguide (cylinder) is generally used that has a defined length and is attached directly under the treatment head. During the treatment, the bottom side (free or terminal end) of the electron waveguide (cylinder) is placed directly onto the skin of the animal. In order to ensure that even animals having a large abdominal circumference, such as, for example, horses, can be appropriately irradiated, it is possible to configure the distance between the support medium (support structure) and the source of emissions (e.g., waves, such as X-rays, or particles, such as electrons) as large as possible, that is, to lower the tabletop as deeply as possible.

Due to the base frame having, for example, a maximum height over the ground of 150 mm, at least in the region of the front end, it is possible in particular to allow a treatment head of a conventional medical linear accelerator to rotate 360 degrees about a longitudinal axis around the protruding (cantilevered) table region of the tabletop. Thus, radio-oncological treatments on the large animal are possible using known linear accelerators, despite the fact that the treatment head in this case sweeps just above the ground during a rotation. In particular, by using a large animal treatment table according to this aspect of the present disclosure, it is thus possible to be able to carry out a so-called rotation irradiation or stereotactic irradiation on the animal, in particular even in regions that are not reachable from above the tabletop.

In a further exemplary embodiment of the present disclosure, the tabletop of the large animal treatment table includes, on at least one longitudinal edge, at least one plug-in option for a table-extension element and/or a fixing device, using which, for example, limbs of a large animal can be held in desired position using belts or tension straps.

To simplify the construction it is advantageous in particular if no specific axis of rotation and corresponding bearing need to be provided to achieve the inclination of the tabletop. In an exemplary embodiment of this aspect of the disclosure, it is thus expedient to rotatably connect a locking element to the upper bearing in the region of the table's rotational axis and the base frame, so that when the lifting device is lowered the tabletop automatically inclines. In accordance with changing distance between the upper pivot bearing of the locking element and the lower pivot bearing on the base frame in conjunction with the lowering height of the table, the table inclination is adjustable.

As was mentioned above, the lifting device preferably comprises two scissor mechanisms spaced from each other in the transverse direction. These scissor mechanisms are height adjustable, e.g., using a common motor and an associated transmission. For this purpose the two scissor mechanisms preferably include an upper support that is rotatably supported on a stationary axis of rotation with the tabletop on a respective rear end of the scissor mechanism. A front end of the respective scissor mechanism is slidably connected to the support.

In a further exemplary embodiment of the present disclosure, the two lower ends of the scissor mechanisms are preferably configured such that a lower rear end of the scissor mechanism is rotatably supported with the base frame and a front lower end is slidably supported on the respective support of the base frame. In this case, large load forces are readily supportable, and despite these large loads, the tabletop can be adjusted in height. Furthermore, a free space is provided both between the base frame or the respective base frame parts and the two lifting mechanisms, so that a completely free (empty) intermediate space is present below the tabletop between the two scissor mechanisms. This free (empty) intermediate space makes it possible to move the tabletop over an existing table of a computer tomography system or the like.

For improved support of differently sized animals, the tabletop is preferably provided with at least one plug-in option for a table-extension element on at least one longitudinal edge and/or side edge.

In a further exemplary embodiment, one or more rollers are motor-driven in order to be able to move the large animal treatment table more easily.

### BRIEF DESCRIPTION OF THE DRAWINGS

For further explanation and for better understanding of the present disclosure, exemplary embodiments are illustrated in the following drawings and are described in more detail below.

FIG. 1 is a schematic oblique view from above of a large animal treatment table according to the present disclosure.

FIG. 2 is a side elevational view of the large animal treatment table of FIG. 1.

FIG. 3 is a side elevational view of the large animal treatment table of FIG. 2 with a tabletop in a lowered and inclined position.

FIG. 4 is a schematic oblique view from above of the large animal treatment table of FIG. 3.

FIG. 5 is an exploded view of the tabletop of the large animal treatment table of FIG. 1 with various attachable accessories (attachment parts).

FIG. 6 is an oblique view of the treatment table of FIG. 5 with the attached accessories.

FIG. 7 is a front elevational view schematically illustrating the large animal treatment table of FIG. 1 with its tabletop in a maximally lowered position.

FIG. 8 illustrates the large animal treatment table of FIG. 7 with its tabletop in a maximally raised position

FIG. 9 is a top plan view of the large animal treatment table according to the disclosure having various accessories (attachment parts).

FIG. 10 is a front elevational view of the large animal treatment table of FIG. 1, in which the table has been positioned (disposed) within an application region of a treatment head of a commercially available medical linear accelerator.

FIG. 11 is a perspective side view of the large animal treatment table and the treatment head of the linear accelerator, as shown in FIG. 10.

FIG. 12 is a perspective view similar to FIG. 11 from another side in which the treatment head is located below the tabletop of the large animal treatment table of FIG. 1.

### DETAILED DESCRIPTION

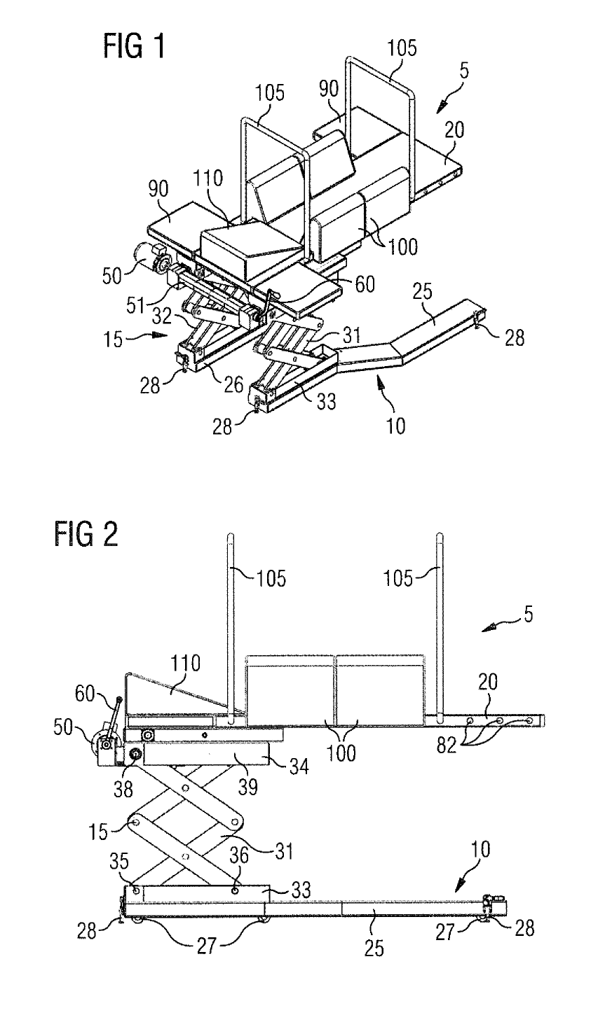

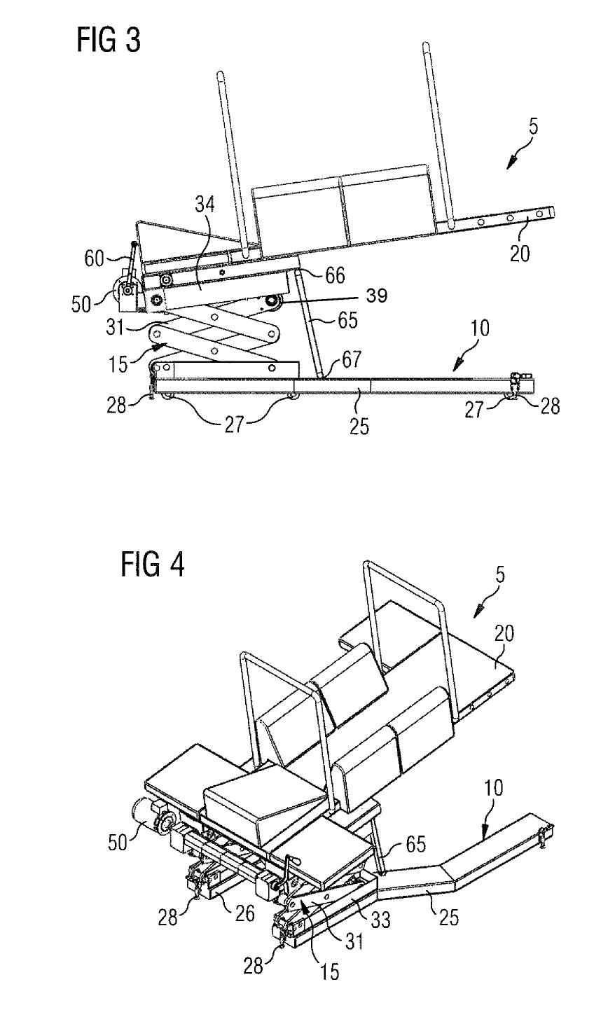

A first exemplary, non-limiting embodiment of a large animal treatment table 5 according to the present disclosure is shown in FIGS. 1 to 4. This large animal treatment table 5 comprises a base frame 10, a lifting device (lift) 15, and a tabletop 20. The tabletop 20 is connected to the base frame 10 via the lifting device 15. As can be seen in particular from FIGS. 1 and 3, these three basic elements of the large animal treatment table 5 are disposed with respect to one another such that, in a side view, they have an asymmetric, or more specifically, a C-shaped contour or outline.

The base frame 10 comprises two spaced-apart chassis parts (feet) 25, 26 which are each equipped with rollers 27 for rolling the table 5 on the floor. In the present embodiment, three rollers 27 are provided on each chassis part, namely, a first roller 27 at the rear end, a second roller 27 in the central region, and a third roller 27 in the front region of the respective chassis part 25, 26. The two chassis parts 25, 26 are spaced from each other by a clear (empty) inner width. As can be seen in FIGS. 1 and 4, a front region of each chassis part 25, 26 is configured (shaped) with a greater clear (wider empty) inner width than at a rear region. In the front region the two mutually inverse opposing chassis parts 25, 26 thereby form a large clear (empty) inner width, which is greater than a clear (empty) inner width at the rear end. This design has the effect of providing greater stability of the entire large animal treatment table 5.

In the present exemplary embodiment the chassis parts 25, 26 are configured in a three-part manner. That is, two straight parts are connected to each other by an outwardly angled central part. At the respective rear end of each chassis part 25, 26, as well as on a front end thereof, locking devices 28 are respectively disposed and may be configured, for example, such that a rubber part may be pressed onto the ground when actuated by a user's foot, thereby increasing friction and restricting movement of the table 5 relative to the floor. The locking devices 27, 28 may be configured in a manner that is common in the market (art).

The lifting device 15 preferably comprises two scissor mechanisms (first and second lifts) 31, 32, which are respectively connected to the chassis parts 25, 26. The right scissor mechanism 31 preferably comprises a lower support 33 and an upper support 34. As shown in FIG. 2, the lower support 33 comprises a rear lower bearing 35, to which a part of the scissor mechanism is rotatably connected. A sliding or rolling bearing 36 is connected to the other lower part of the scissor mechanism 31 and thus slides or rolls in the longitudinal direction on the rear part of the chassis part 25.

The upper support 34 comprises an upper rear bearing 38 and an upper front sliding or rolling bearing 39. The upper rear bearing 38 is rotatably connected to one upper part of the scissor mechanism 31. The upper front sliding or rolling bearing 39 is connected to another upper part of the scissor mechanism 31 and rolls on the underside of the tabletop or a plate inserted therebetween. The left scissor mechanism 32 is preferably configured in the same manner as the right scissor mechanism 31.

In FIG. 3, the actual detailed structure and the transmission of the height adjustment are only suggested and partially visible. Referring to FIG. 1, an electric motor 50 is connected via a transmission shaft 51 to a (not shown) spindle that changes the distance of the two parts of the upper scissor mechanism relative to each other and thus causes the scissor mechanism 31 to lift or lower the upper support 34. Both scissor mechanisms 31, 32 are preferably connected to the same transmission shaft of the electric motor in order to achieve a uniform lowering and lifting of the tabletop 20.

As can be seen in particular from FIGS. 1 and 3, a crank 60 is also provided, using which the transmission shaft 51 can be rotated by hand and thus the height of the tabletop 20 can be finely adjusted, or in the case of a failure of the electric motor, enable the tabletop 20 to be raised and lowered manually.

As can be seen in particular from FIGS. 3 and 4, the tabletop 20 can be inclined. In the present exemplary embodiment, the inclination is adjusted by adjusting the height of the tabletop 20 using the electric motor 50 or the hand crank 60. For this purpose a locking bracket (bar, rod) 65 is pivotably (hingedly) connected to an underside of the tabletop 20 and to the respective chassis part 25, 26 at an upper pivot bearing point 66 and a lower pivot bearing point 67. Due to the rigidity (stiffness) of the locking bracket 65, the distance between the upper pivot bearing point 66 and the lower pivot bearing point 67 is fixed by this connection. If as shown, for example, in FIG. 3, a lowering of the tabletop 20 is now effected, the front part in the region of the upper support 34 is not lowered and instead only the rear part is lowered. This inclination results because the respective front regions of the scissor mechanisms 31, 32 have a sliding or rolling bearing as can be seen in particular in FIG. 3, where the front rolling bearing 39 of the scissor mechanism 31 is brought (lowered) out of the upper support 34 due to the inclination effected by the locking bracket 65. The greater the lowering of the table 20 with inserted locking bracket 65, the greater the inclination of the tabletop 20 becomes.

The locking bracket 65 is preferably configured to be removable or, for example, to be pivotally attached to the upper pivot bearing point 66, so that, when not in use, it can be pivotally fixed to the underside of the tabletop 20 such that no locking effect is produced. When inclination of the tabletop 20 is desired, the locking bracket 65 is simply pivoted downward and connected to the lower pivot bearing point 67.

The locking bracket 65 can also be configured only as a bar (rod) or can have another shape.

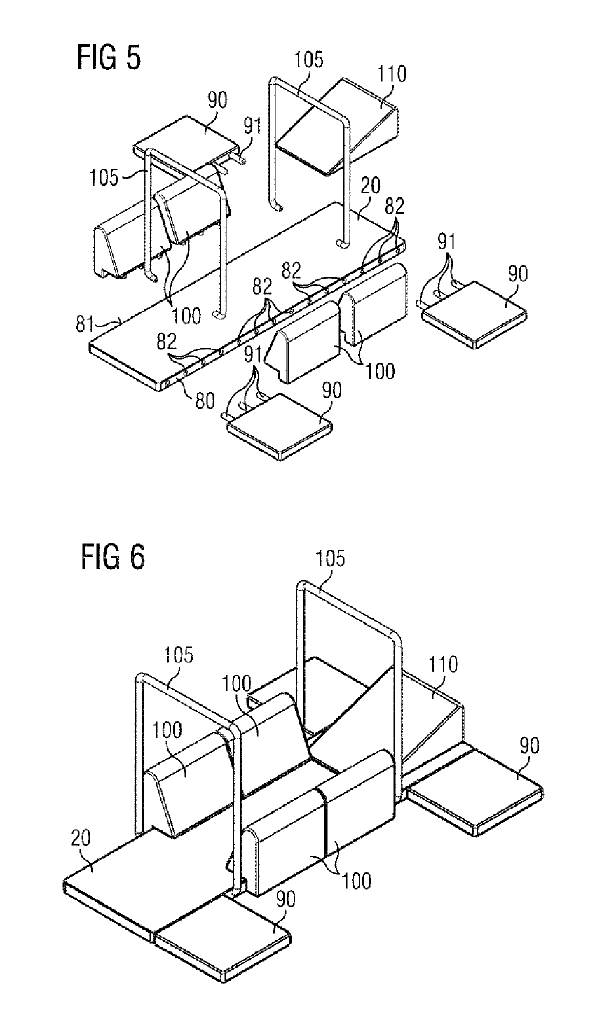

Next, the design of the tabletop 20 will be described in further detail with reference to FIGS. 5 and 6. Preferably, the tabletop 20 is made of CRP and/or other radiolucent materials and includes a row of holes (receivers) 82 on the two longitudinal edges 80, 81. These holes 82 serve as plug openings for complementarily configured pins 91 of various accessories (attachment parts) such as tabletop extensions 90, side-wall parts 100, and fixing supports 105 as well as possibly a head part (head rest) 110.

In FIG. 6 the tabletop 20 is shown with the various accessories (attachment parts) 90, 100, 105 as well as the head part 110. Here these accessories (attachment parts) 90, 100, 105 are inserted onto (into) the tabletop using their plug-pins. Owing to the various rows of holes, the accessories can be placed individually (in a custom manner) depending on requirements (e.g., size, condition, procedure to be performed, etc.) of the animal. The fixing supports 105 serve to provide a fixing option for limbs of horses or other large animals. Here, for example, individual limbs are fixed using belts or straps.

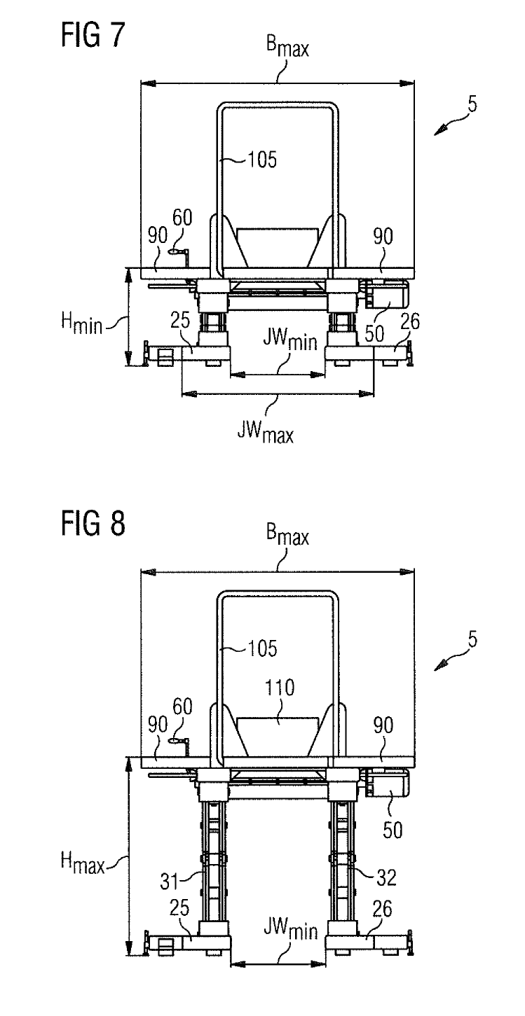

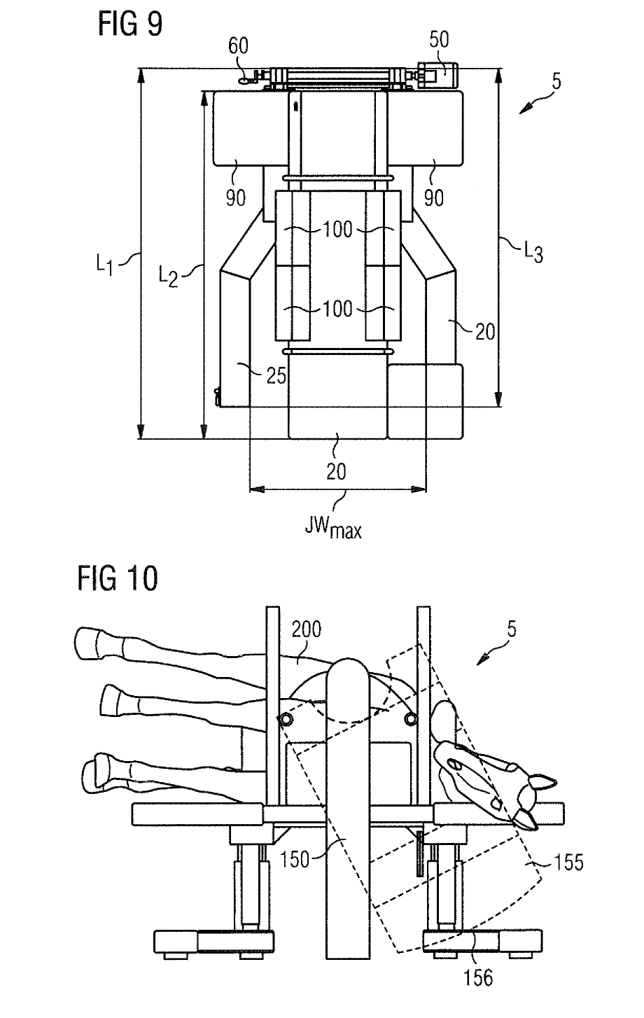

In FIGS. 7 to 9, the dimensions for advancing (moving) the large animal treatment table 5 over tables of existing computer tomography devices or the like are more clearly visible. The clear (empty) inner width JWₘᵢₙ between the two chassis parts 25, 26 at the narrowest point in the present exemplary embodiment is 700 mm. In the front region, the clear (empty) inner width JWₘₐₓ is 800 mm. Furthermore, in the present exemplary embodiment, the minimum lifting height Hₘᵢₙ is approximately 715 mm (see FIG. 7) and the maximum lifting height Hₘₐₓ is approximately 1470 mm (see FIG. 8). The lifting heights Hₘᵢₙ or Hₘₐₓ are measured from the upper edge (surface) of the tabletop 20 to the ground (floor). When the table extensions 90 are attached, the table width Bₘₐₓ is 2000 mm. The overall length L1 of the treatment table is approximately 3000 mm and the table length L2 is approximately 2000 mm. The chassis parts 25, 26 each have a length L3 of approximately 2750 mm.

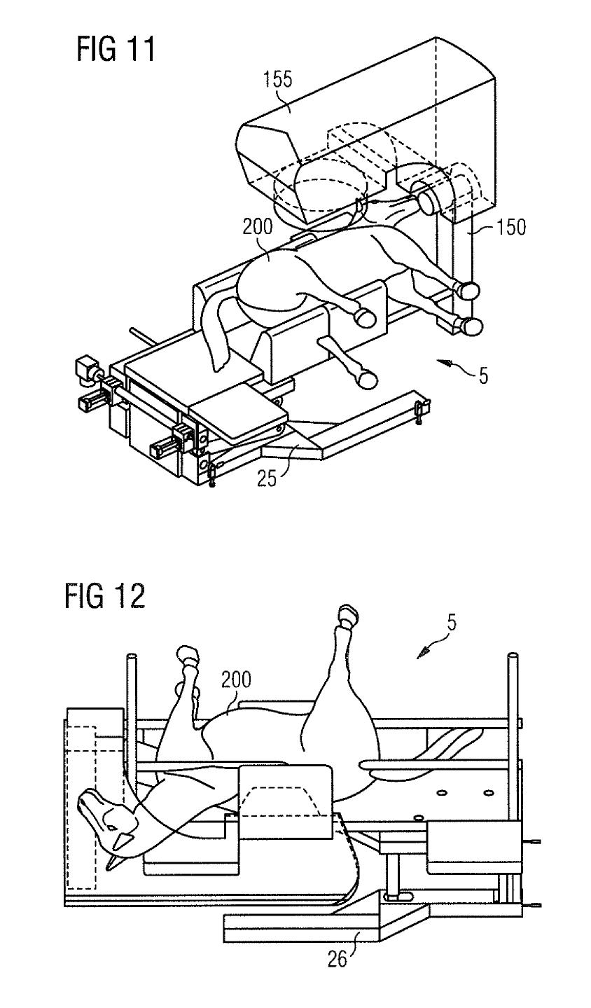

FIGS. 10 to 12 show how the exemplary large animal treatment table 5 is usable in connection with a medical linear accelerator having a treatment head 155. Here the treatment head 155 of the medical linear accelerator is rotatably supported on a column 150. Owing to the design of the large animal treatment table 5 having the C-shaped outline in the side view and large opening in the front region of the chassis including the chassis parts 25, 26, it is possible to move the large animal treatment table 5, with a horse 200 or another animal supported thereon, up to the medical linear accelerator, even if the medical linear accelerator includes its own table. Furthermore it is even possible to rotate the treatment head 155, as illustrated in FIGS. 10 and 12, below (underneath) the treatment table 5 in order to carry out certain treatments. As shown in FIG. 10, the outermost side 156 of the treatment head 155 sweeps just over the ground and the front ends of the two chassis parts 25, 26.

Computer tomography devices (CT scanners) that are combinable (usable together) with the disclosed large animal treatment table are distributed, for example, by Siemens AG, General Electric, Philips, and Toshiba. Medical (charged particle) linear accelerators that are suitable for carrying out treatments using the exemplary large animal treatment table are distributed under the names Varian® Clinac® (with and without OBI), in particular of the design Clinac® 2100, Clinac® 2100 C/D or Clinac® iX, TrueBeam®, Elekta® Synergy®, and Elekta® Versa™.

Finally it should be noted that large animal treatment tables of the design disclosed here may be statically configured such that animals having a body weight of up to 500 kg or 800 kg or in particular even up to 1,000-1,500 kg can be supported on the tabletop. In the present case treatment tables are to be subsumed under the term “large animal treatment table” that are designed for loads of at least 300 kg or 500 kg, but preferably can support loads of up to 1000 kg, 1300 kg or even 1500 kg.

The terms “roughly,” “about,” “approximately,” “essentially,” or “generally” used here, which are used in connection with a measurable value such as, for example, a parameter, a quantity, a shape, a time duration, or the like, or also explicit value indications include deviations or fluctuations of ±10% or less, preferably ±5% or less, further preferably ±1% or less, and further preferably ±0.1% of the respective value, provided these deviations are still technically practicable in the implementation of the disclosed invention in practice. It is expressly indicated that the value to which the term “approximately” refers is explicitly and specifically disclosed as such. The indication of ranges by initial and final values comprises all those values and fractions of those values that are enclosed by the respective range as well as its initial and final values.

Representative, non-limiting examples of the present invention were described above in detail with reference to the attached drawings. This detailed description is merely intended to teach a person of skill in the art further details for practicing preferred aspects of the present teachings and is not intended to limit the scope of the invention. Furthermore, each of the additional features and teachings disclosed above may be utilized separately or in conjunction with other features and teachings to provide improved large animal treatment tables.

Moreover, combinations of features and steps disclosed in the above detailed description may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims. In addition, all value ranges or indications of groups of entities are intended to disclose every possible intermediate value or intermediate entity for the purpose of original written disclosure, as well as for the purpose of restricting the claimed subject matter.

Additional representative embodiments of the present disclosure, include but are not limited to:

1. Large animal treatment table (5) including:
a movable base frame (10) which, viewed in the longitudinal axis direction, includes a front end and a rear end, and at least in the region of the front end comprises a divided chassis including a right chassis part (25) and a left chassis part (26) spaced from the right chassis part (25);
a lifting device (15) that includes a lower support (33), using which the lifting device (15) is disposed in the region of the rear end of the base frame (10), and includes an upper support (34);
a tabletop (20) connected to the upper support (34) of the lifting device (15) such that the tabletop (20) has a freely projecting (cantilevered) table region, and in a side view the base frame (10) the lifting device (15) and the tabletop (20) have a C-shaped outline, so that in cooperation with the divided chassis (25, 26) the tabletop is movable over a table of a conventional treatment- or diagnosis-device such as, for example, a computer tomography device, and additionally or alternatively a treatment head of a linear accelerator is rotatable about the protruding table region.

2. Large animal treatment table according to embodiment 1, wherein viewed in the longitudinal direction the lifting device (15) comprises a left lifting part (31) and a right lifting part (32), respectively, between which a free space is present that in particular makes possible driving over (advancing over) an existing treatment table of a computer tomography device or linear accelerator.

3. Large animal treatment table according to embodiment 2, wherein a motor (50) adjusts both the right and the left lifting part (31, 32) in height via a mechanical transmission (51).

4. Large animal treatment table according to embodiment 2 or 3, wherein the right and left lifting parts each comprise a scissor mechanism (31, 32).

5. Large animal treatment table (5) according to one of the preceding embodiments, wherein the base frame (10) has a greater width at the front end than at the rear end.

6. Large animal treatment table according to one of the preceding embodiments, wherein the tabletop (20) is inclinable.

7. Large animal treatment table according to embodiment 6, wherein a rigid locking element (60) is rotatably connectable at one end to the upper support (34) and by the other end to the base frame (10), so that the tabletop (20) automatically inclines when the lifting device (15) is lowered.

8. Large animal treatment table according to one of the preceding embodiments, wherein the upper support (34) of the lifting device (15) is rotatably supported about a transverse axis with the table (20) at a rear upper bearing and is slidingly supported with the tabletop (20) at a rear lower bearing.

9. Large animal treatment table according to one of the preceding embodiments, wherein the lower support (33) of the lifting device (15) is rotatably supported about a transverse axis with the base frame (10) at a rear bearing and is slidingly supported at the base frame (10) at a front bearing (36).

10. Large animal treatment table (5) according to one of the preceding embodiments, wherein the right chassis part (25) and the left chassis part (26) are each provided with rollers (27) and preferably at least in the region of the front end define a free opening (clearance) having a clear inner width (JW) of at least 700 mm.

11. Large animal treatment table (5) according to one of the preceding embodiments, wherein at least in the region of the front end the base frame (10) has a maximum height (Hₘₐₓ) over the base of 150 mm.

12. Large animal treatment table according to one of the preceding embodiments, wherein the tabletop (20) is lowerable to a minimum height (Hₘᵢₙ) of 715 mm.

13. Large animal treatment table according to one of the preceding embodiments, wherein the tabletop (20) is liftable to a maximum height (Hₘₐₓ) of 1400 mm.

14. Large animal treatment table according to one of the preceding embodiments, wherein on at least one longitudinal edge (80, 81) and/or side edge the tabletop (20) includes at least one plug-in option (82) for one or more accessories (attachment parts) such as table extension elements (90), side wall parts (100), or fixing supports (105).

15. Large animal treatment table according to one of the preceding embodiments, wherein in addition to the motor (50) for height adjustment of the tabletop (20), a crank (60) to be actuated by hand is provided for height adjustment of the tabletop (20).

We claim:

1. A large animal treatment table comprising:
a movable base frame having a front portion with a front end and a rear portion with a rear end, the front end being spaced from the rear end in a longitudinal direction and the front portion being separated from the rear portion by a transverse center plane perpendicular to the longitudinal direction, the front portion comprising a right front frame part on a first side of a longitudinal centerline and a left front frame part on a second side of the longitudinal centerline and spaced from the right front frame part by a first gap;
at least one lift disposed entirely between the rear end and the transverse center plane, the at least one lift having a lower support mounted on the base frame and an upper support; and
a tabletop connected to the upper support of the at least one lift and having a front portion and a rear portion separated by the center plane, the front portion of the tabletop being cantilevered over the first gap such that, in a side view, the base frame and the at least one lift and the tabletop have a C-shaped profile.

2. The large animal treatment table according to claim 1, wherein the base frame and the tabletop are configured such that:
the cantilevered front portion is movable over a table of a treatment or diagnosis device, and
an emitter head of a medical linear accelerator is rotatable around the cantilevered front portion.

3. The large animal treatment table according to claim 2, wherein:
the rear portion of the frame includes a left rear frame part on the first side of the longitudinal centerline and a right rear frame part on the second side of the longitudinal centerline and separated from the left rear frame part by a second gap,
the at least one lift includes a first lift connected between the left rear frame part and the tabletop and a second lift connected between the right rear frame part and the tabletop, and
when viewed longitudinally from the rear, the treatment table has an inverted U-shaped profile configured to allow the treatment table to move over the table of the treatment or diagnosis device.

4. The large animal treatment table according to claim 3, including a motor operably connected to the first lift and to the second lift by a mechanical transmission,
wherein the first lift comprises a first scissor lift and the second lift comprises a second scissor lift.

5. The large animal treatment table according to claim 1, wherein the front portion of the movable base frame has a greater width than the rear portion of the base frame.

6. The large animal treatment table according to claim 5, wherein the first gap is wider than the second gap.

7. The large animal treatment table according to claim 1, wherein the tabletop is configured to tilt relative to the movable base frame.

8. The large animal treatment table according to claim 7, including a rod having a first end pivotably connectable to the upper support and a second end pivotably connectable to the base frame such that the front portion of the tabletop rises when the at least one lift is lowered.

9. The large animal treatment table according to claim 1, wherein the upper support of the at least one lift has a first bearing configured to support the upper support for rotation about a transverse axis and the lower support of the at least one lift has a second bearing configured to slidingly support the lower support relative to the base frame.

10. The large animal treatment table according to claim 9, wherein the lower support of the at least one lift is rotatably supported on the base frame by a third bearing.

11. The large animal treatment table according to claim 1, wherein the right front frame part and the left front frame part are each provided with rollers and wherein the first gap has a width of at least 700 mm.

12. The large animal treatment table according to claim 1, wherein the right front base frame has a top surface located no more than 150 mm above a surface on which the large animal treatment table is supported.

13. The large animal treatment table according to claim 1, wherein the at least one lift is configured to lower the tabletop to a minimum height (Hₘᵢₙ) of 715 mm above a surface on which the large animal treatment table is supported.

14. The large animal treatment table according to claim 13, wherein the at least one lift is configured to raise the tabletop to a maximum height (Hₘₐₓ) of 1400 mm above the surface on which the large animal treatment table is supported.

15. The large animal treatment table according to claim 1, wherein at least one longitudinal edge and/or side edge the tabletop includes at least one receiver configured to connect one or more accessories to the tabletop.

16. The large animal treatment table according to claim 1, including a hand crank configured to raise and lower the tabletop relative to the base frame.

17. The large animal treatment table according to claim 4, wherein:
the front portion of the base frame has a greater width than the rear portion of the base frame,
the first gap is wider than the second gap,
the tabletop is configured to tilt relative to the base frame,
a rod has a first end rotatably connectable to the upper support and a second end connectable to the base frame such that the front portion of the tabletop rises when the at least one lift is lowered,
the right front frame part and the left front frame part are each provided with rollers,
the right front base frame has a top surface located no more than 150 mm above a surface on which the large animal treatment table is supported, and
the at least one lift is configured to lower the tabletop to a minimum height (Hₘᵢₙ) of 715 mm above a surface on which the large animal treatment table is supported and to raise the tabletop to a maximum height (Hₘₐₓ) of 1400 mm above the surface.

18. A large animal treatment table comprising:
a movable base frame comprising a first longitudinally extending foot having a front half separated from a rear half by a transverse center plane and a second longitudinally extending foot having a front half spaced from a rear half by the center plane, the first foot being separated from the second foot by a first gap;
a first lift mounted on the first foot and located entirely on the rear side of the center plane and a second lift mounted on the second foot and being located entirely on the rear side of the center plane and separated from the first lift by a second gap; and
a tabletop connected to the first lift and to the second lift such that at least a front half of the tabletop is cantilevered over the first gap,
wherein the first longitudinally extending foot, the first lift and the tabletop have a C-shaped profile in a side view and the first longitudinally extending foot, the first lift, the tabletop, the second lift and the second longitudinally extending foot have an inverted U-shaped profile in an end view such that the tabletop is movable over a table of a treatment or diagnosis device.

19. The large animal treatment table according to claim 18, including a motor operably connected to the first lift and to the second lift by a mechanical transmission.

20. The large animal treatment table according to claim 19, wherein the first lift comprises a first scissor lift and the second lift comprises a second scissor lift.
